(19) United States
(12) Patent Application Publication
(10) Pub. No.: US 2024/0180817 A1
(43) Pub. Date: Jun. 6, 2024

(54) PLANT FERMENTED LIQUID, PLANT FERMENTED PRODUCT PRODUCED USING GLUCONOBACTER THAILANDICUS STRAIN AND USE THEREOF

(71) Applicants: LG HOUSEHOLD & HEALTH CARE LTD., Seoul (KR); KELP INC., Sapporo (JP)

(72) Inventors: Mirim KIM, Seoul (KR); Munemitsu Fukushi, Sapporo (JP); Haemin Jung, Seoul (KR); Heqing Cui, Seoul (KR)

(73) Assignees: LG HOUSEHOLD & HEALTH CARE LTD., Seoul (KR); KELP INC., Sapporo (JP)

(21) Appl. No.: 18/515,103

(22) Filed: Nov. 20, 2023

(30) Foreign Application Priority Data
Dec. 5, 2022 (KR) 10-2022-0167828

(51) Int. Cl. A61K 8/99; A61Q 19/00; C12N 1/20

(52) U.S. Cl. CPC A61K 8/99; A61Q 19/00; C12N 1/205; C12R 2001/01

(57) ABSTRACT

The present invention relates to a Gluconobacter thailandicus strain deposited under Accession No. KCTC 15086BP; a method for producing plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger; a method for producing a plant fermented product by fermenting the plant fermented liquid with an acetic acid bacterial strain; a cosmetic composition, a quasi-drug composition, or a food composition comprising the plant fermented product as an active ingredient.

Since the plant fermented liquid, the Gluconobacter thailandicus strain, a culture of the strain, or a fermented product of the strain has an excellent skin improvement effect, it can be widely used as cosmetic compositions, quasi-drug compositions, and food compositions for skin improvement. Accordingly, various industrial applications such as cosmetics, quasi-drugs, and foods can be expected.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 15, 2023, is named “2023-12-11-SeqListing-4698-0133PUS1” and is 2,883 bytes in size. The sequence listing contained in this .XML file is part of the specification and is hereby incorporated by reference herein in its entirety.

### TECHNICAL FIELD

The present invention relates to a Gluconobacter thailandicus strain deposited under Accession No. KCTC 15086BP; a method for producing plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger; a method for producing a plant fermented product by fermenting the plant fermented liquid with an acetic acid bacterial strain; and a cosmetic composition, a quasi-drug composition, or a food composition comprising the plant fermented product as an active ingredient.

### BACKGROUND ART

Cosmetic ingredients are generally known to cause various skin problems such as inflammation, pimples, edema, etc., (Maibach, H. I., Contact Dermatitis, 6. 369-404, 1980). In addition, various substances used as raw materials for functional cosmetics also have a problem of low stability or insignificant effects on the skin.

In one example, retinoids, adenosine, animal placenta-derived proteins, chlorella extracts, etc. are known as cosmetics for improving skin wrinkles and elasticity. Retinol, which is the most widely known, has limitations in usage due to safety issues such as irritation when applied to the skin, and chlorella extracts, etc. have an insignificant effect, and thus, it is difficult to substantially expect skin elasticity enhancement and wrinkle improvement effects.

A drug that acts to remove an inflammatory source to eliminate the inflammation and to reduce vital reaction and symptoms is called an anti-inflammatory agent. Thus far, substances used for the purpose of anti-inflammation include non-steroids such as flufenamic acid, ibuprofen, benzydamine, indomethacin, etc., and steroids such as prednisolone, dexamethasone, etc., but the use thereof is limited because most of these substances have safety issues on the skin or stability issues when containing cosmetics.

Recently, many studies have been conducted on cosmetic compositions containing plant extracts of natural materials, which are effective in improving skin conditions or preventing skin aging or skin damage, as they have excellent skin soothing, moisturizing, anti-inflammatory, or whitening effects as active ingredients. However, when plant extracts are used alone, skin improvement effects may be insignificant in most cases, and there are many cases where they cannot be used in various formulations because a large amount may be used for application in order to to exert efficacy in products.

Under such circumstances, the present inventors have conducted research on raw materials that are safer and have excellent skin improvement effects from natural resources, and have confirmed that plant fermented liquid fermented for more than 5 years in the natural environment of Hokkaido and the fermented product obtained by secondary fermentation of the plant fermented liquid using Gluconobacter thailandicus showed significantly excellent skin improvement effects even at a lower content compared to the one-year fermented liquid, thereby completing the present invention.

### DISCLOSURE

### Technical Problem

It is one object of the present invention to provide a Gluconobacter thailandicus strain deposited under Accession No. KCTC 15086BP.

It is another object of the present invention to provide a cosmetic composition for improving skin, including plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient.

It is still another object of the present invention to provide a quasi-drug composition for improving skin, including plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient.

It is yet another object of the present invention to provide a food composition for improving skin, including plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient.

It is even another aspect of the present invention to provide a method for producing a plant fermented product, including: fermenting plant fermented liquid by an acetic acid bacterial strain, a culture of the strain, a fermented product of the strain, or a combination thereof.

It is further another aspect of the present invention to provide a plant fermented product produced by the method above.

It is still further another aspect of the present invention to provide a method for improving skin, including: applying a composition containing any one or more among the Gluconobacter thailandicus strain deposited under Accession No. KCTC 15086BP, a culture of the strain, and a fermented product of the strain to a subject.

It is still further another aspect of the present invention to provide a method for improving skin, including: applying a composition containing plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient to a subject.

### Technical Solution

Hereinafter, the present invention will be described in detail. Meanwhile, each description and embodiment disclosed herein can be applied to other descriptions and embodiments, respectively. That is, all combinations of various elements disclosed herein fall within the scope of the present invention. Further, the scope of the present invention is not limited by the specific description described below.

Additionally, those of ordinary skill in the art may be able to recognize or confirm, using only conventional experimentation, many equivalents to the particular aspects of the invention described herein. Furthermore, it is also intended that these equivalents be included in the present invention.

One aspect of the present invention provides a Gluconobacter thailandicus strain deposited under Accession No. KCTC 15086BP.

As used herein, the term “Gluconobacter thailandicus” refers to acetic acid bacteria first isolated in Thailand. The characteristics of Gluconobacter thailandicus vary depending on the growth region and natural environmental conditions, and thus, the activity of the strain and its fermented product may vary, and the Gluconobacter thailandicus of the present invention may be derived from a Japanese apricot enzyme solution, but it not limited thereto.

The strain of the present invention, a culture of the strain, or a fermented product of the strain may have a skin improvement effect.

As used herein, the term “strain” refers to a set of individuals derived from a single cell, which proliferates through asexual reproduction and has homogeneous genetic characteristics, and several strains (genetic variants) with different genetic characteristics may exist within one species. In the present invention, the strain refers to Gluconobacter thailandicus, and may specifically be Gluconobacter thailandicus deposited under Accession No. KCTC 15086BP, but is not limited thereto.

As used herein, the “culture” refers to a product obtained by culturing the strain in a medium. For example, the culture of the present invention may include components remaining in the culture medium of Gluconobacter thailandicus after harvesting the strain.

The culture may be the whole culture of the Gluconobacter thailandicus strain, a supernatant of the culture, a lysate of the culture, a fraction thereof, etc. In particular, the supernatant may be obtained by centrifuging the culture of the strain, the lysate may be obtained by physical treatment or sonication of the strain, and the fraction may be obtained by conducting centrifugation, chromatography, etc. on the culture, supernatant, lysate, etc.

The medium and other culture conditions used for culturing the strain of the present invention may be any medium used for conventional culturing of microorganisms belonging to the genus Gluconobacter without any particular limitation. Specifically, the strain of the present invention may be cultured under aerobic or anaerobic conditions in a conventional medium containing an appropriate carbon source, nitrogen source, phosphorus source, inorganic compound, amino acid, and/or vitamin, while adjusting temperature, pH, etc.

In the present invention, the carbon source may include carbohydrates, such as glucose, fructose, sucrose, maltose, etc.; sugar alcohols, such as mannitol, sorbitol, etc.; organic acids, such as pyruvic acid, lactic acid, citric acid, etc.; amino acids, such as glutamate, methionine, lysine, etc., but is not limited thereto. Additionally, the carbon source may include natural organic nutrients such as starch hydrolysate, molasses, blackstrap molasses, rice bran, cassava, sugar cane molasses, corn steep liquor, etc., and carbohydrates such as glucose and sterilized pretreated molasses (i.e., molasses converted to reducing sugar) may be used. In addition, various other carbon sources in an appropriate amount may be used without limitation. These carbon sources may be used alone or in a combination of two or more kinds.

The nitrogen source may include inorganic nitrogen sources, such as ammonia, ammonium sulfate, ammonium chloride, ammonium acetate, ammonium phosphate, ammonium carbonate, ammonium nitrate, etc.; and organic nitrogen sources, such as amino acids, peptone, NZ-amine, meat extract, yeast extract, malt extract, corn steep liquor, casein hydrolysate, fish or decomposition product thereof, defatted soybean cake or a decomposition product thereof, etc. These nitrogen sources may be used alone or in a combination of two or more kinds, but are not limited thereto.

The phosphorus source may include monopotassium phosphate, dipotassium phosphate, or corresponding sodium-containing salts, etc. Examples of the inorganic compounds may include sodium chloride, calcium chloride, iron chloride, magnesium sulfate, iron sulfate, manganese sulfate, calcium carbonate, etc.

Additionally, amino acids, vitamins, and/or appropriate precursors may be included in the medium. Specifically, L-amino acids, etc. may be added to the culture medium of the strain. Specifically, glycine, glutamate, and/or cysteine may be added, and if necessary, L-amino acids such as lysine may be further added, but are not necessarily limited thereto.

The medium or precursors may be added to a culture in a batch or continuous manner, but is not limited thereto.

In the present invention, the pH of the culture may be adjusted by adding a compound such as ammonium hydroxide, potassium hydroxide, ammonia, phosphoric acid, sulfuric acid, etc. during the culturing of the strain in an appropriate manner. In addition, bubble formation may be prevented during culturing using an antifoaming agent such as fatty acid polyglycol ester. Further, oxygen gas or a gas containing oxygen may be injected to the culture order to maintain aerobic conditions of the culture; or nitrogen gas, hydrogen gas, or carbon dioxide may be injected, or gas may not be injected, to maintain anaerobic or microaerobic conditions.

As used herein, the term “fermentation” means that microorganisms are not putrefactive during the process of decomposing organic matter using their own enzymes. Fermentation reaction and decay reaction proceed by similar processes, but when decomposition produces useful substances, it is called fermentation, and when odorous or harmful substances are produced, it is called decay.

In the present invention, the method for obtaining the fermented product from the strain is not particularly limited, and may be obtained according to a method commonly used in the art or similar fields.

In the present invention, the fermented product obtained from the strain may include not only the fermented material itself, but also all kinds of materials including fermented products produced from the strain, such as a culture medium of the strain in which the strain and the culture coexist, a fermented product obtained by filtering the strain from the culture medium, a fermented product obtained by sterilizing the strain from the culture medium and filtering the same, an extract obtained by extracting the fermented product or a culture medium containing the same, a diluted solution obtained by diluting the fermented product or an extract thereof, a dried product obtained by drying the fermented product or an extract thereof, and a lysate obtained by collecting and lysing the cells of the strain, etc.

In the present invention, the strain may include cells, dried cells, lysates, etc. of the Gluconobacter thailandicus strain. In particular, the dried cells may be spray-dried bacteria, freeze-dried bacteria, vacuum-dried bacteria, drum-dried bacteria, etc., and the lysate may be a product obtained by breaking the cell walls of the strain itself by chemical or physical force.

Another aspect of the present invention provides a cosmetic composition for improving skin, including plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient.

As used herein, the “plant fermented liquid” may be a product obtained by aging and fermenting okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, ginger, etc., but is not limited thereto.

In the present invention, the plant may mean a specific part of the plant, such as the root, leaf, flower, stem, etc., or may mean the whole plant.

In the present invention, the above 55 kinds of plants were selected, and plant fermented liquids were produced by fermenting for 5 years or more or 8 years or more, 10 years or more or 20 years or more, specifically 5 years to 20 years, 8 years to 15 years, or 9 years to 11 years in the natural environment of Hokkaido where the average temperature is about 10° C. to 40° C. and the humidity is about 50% to 80%, specifically, the average temperature is about 20° C. to 30° C., the humidity is about 60% to 70%. In addition, it was confirmed that the plant fermented liquids produced by fermenting for 5 years, 8 years, 10 years, or 20 years or more showed excellent skin improvement effect compared to the plant fermented liquid produced by fermenting for 1 year.

Still another aspect of the present invention provides a cosmetic composition for improving skin, in which plant fermented liquid is fermented by an acetic acid bacterial strain, a culture of the strain, a fermented product of the strain, or a combination thereof.

The “strain”, “culture product”, and “fermentation” are as described above.

As used herein, the term “acetic acid bacteria” are mostly Gram-negative in Gram stain, slightly oval-shaped bacilli, which belong to the genus of bacteria that produce acetic acid using alcohol or lactate as a carbon source, grow aerobically, and do not decompose sugar anaerobically. In the present invention, the acetic acid bacteria may be Gluconobacter thailandicus, specifically Gluconobacter thailandicus deposited under the Accession No. KCTC 15086BP, but the type of strain is not limited as long as it is acetic acid bacteria capable of fermenting plant fermented liquid.

In the present invention, the acetic acid bacteria strain may include cells, dried cells, lysates, etc. of the Gluconobacter thailandicus strain. In particular, the dried cells may be spray-dried bacteria, freeze-dried bacteria, vacuum-dried bacteria, drum-dried bacteria, etc., and the lysate may be a product obtained by breaking the cell walls of the strain itself by chemical or physical force.

In the present invention, the plant fermented liquid may have undergone primary fermentation, or additional fermentation (i.e., secondary fermentation), in which the plant fermented liquid subjected to the primary fermentation is additionally fermented by the strain, a culture of the strain, a fermented product of the strain, or a combination thereof, but is not limited thereto. The additionally fermented plant fermented liquid of the present invention may be used interchangeably with the term plant fermented product or acetic acid bacteria fermented product of plant fermented liquid. The additional fermentation (secondary fermentation) may be performed by inoculating the plant fermented liquid with the strain, a culture thereof, a fermented product thereof, etc., but is not limited thereto. It was confirmed that the plant fermented liquid additionally fermented by the strain, a culture thereof, a fermented product thereof or a combination thereof according to the present invention has much more excellent skin improvement effect.

In the present invention, the method for obtaining the fermented product from the strain is not particularly limited, but acetic acid bacteria fermented products may be obtained by isolating the Gluconobacter thailandicus strain from a Japanese apricot enzyme solution fermented for 4 to 8 months, followed by subjecting the strain to secondary fermentation in the plant fermented liquid. Alternatively, it can be obtained according to a method commonly used in the art or similar fields.

In the present invention, the fermented product obtained from the strain may include not only the fermented material itself, but also all kinds of materials including fermented products produced from the strain, such as a culture medium of the strain in which the strain and the culture coexist, a fermented product obtained by filtering the strain from the culture medium, a fermented product obtained by sterilizing the strain from the culture medium and filtering the same, an extract obtained by extracting the fermented product or a culture medium containing the same, a diluted solution obtained by diluting the fermented product or an extract thereof, a dried product obtained by drying the fermented product or an extract thereof, and a lysate obtained by collecting and lysing the cells of the strain, etc.

As used herein, the term “skin improvement” may be any one or more selected from the group consisting of wrinkle improvement, skin elasticity improvement, skin inflammation improvement, skin soothing, skin moisturization, and skin whitening.

As used herein, the term “improvement” refers to all activities that at least reduce the parameters associated with alleviation or treatment of conditions, e.g., the degree of symptoms, and may include any one or more among wrinkle improvement, skin elasticity improvement, skin inflammation improvement, skin soothing, skin moisturization, and skin whitening.

The skin improvement may be achieved through promoting collagen synthesis, inhibiting NO production, promoting hyaluronic acid synthesis, or inhibiting melanin expression, but is not limited thereto.

Specifically, the present inventors confirmed that the plant fermented liquid and/or the fermented product of Gluconobacter thailandicus KCTC 15086BP had excellent skin improvement effect even with a small amount. More specifically, it was confirmed that the plant fermented liquid and/or the fermented product of Gluconobacter thailandicus KCTC 15086BP were excellent in promoting collagen synthesis, inhibiting NO production, promoting hyaluronic acid synthesis, or inhibiting melanin expression, etc. Therefore, it was confirmed that the plant fermented liquid and the Gluconobacter thailandicus fermented product of the plant fermented liquid exhibited excellent skin improvement effects such as wrinkle improvement, skin elasticity improvement, skin inflammation improvement, skin soothing, skin moisturization, and skin whitening, etc., compared to normal acetic acid bacteria.

As used herein, the term “wrinkle” can be largely divided into facial wrinkles caused by facial expression muscles used to make facial expressions and fine wrinkles caused by overall or localized skin weakness. Specifically, wrinkles may be caused by genes, a decrease in collagen and elastic fibers present in the skin dermis, an external environment, etc. For the purpose of the present invention, wrinkles may be used in the sense of including all facial wrinkles or fine wrinkles, but is not limited thereto.

As used herein, the term “elasticity” refers to the force that tightly holds the skin when the skin is pressed with a finger. The term “elasticity improvement” or “elasticity enhancement” refers to increasing the elasticity of the skin by strengthening the structure of the subcutaneous fat layer of an individual, and may refer to increasing skin elasticity by the composition of the present invention.

As used herein, the term “aging” means a phenomenon in which body structures and functions deteriorate with the time, and the anti-aging of the present invention is not limited to an extent as long as aging is improved, such as preventing, suppressing, delaying aging, etc. Wrinkle improvement and anti-aging in the present invention may be caused by promoting collagen synthesis, but is not limited thereto.

As used herein, the term “collagen” refers to a fibrous protein largely found in most animals, especially mammals, covering almost all connective tissues in the body, such as skin and cartilage, and fibroblasts, the most common cells in the body, produce and secrete collagen. Gelatin, which is widely used in cooking or in the food and pharmaceutical industries, is the product obtained by irreversible hydrolysis of collagen. Since the decrease in collagen causes wrinkles and is the main cause of reducing skin elasticity, synthesis of collagen is essential for improving wrinkles and enhancing elasticity.

In one embodiment of the present invention, it was confirmed that the plant fermented liquid and/or the acetic acid bacteria fermented product of the plant fermented liquid had an excellent collagen synthesis enhancement rate even with a small amount, and based on the result, it was confirmed that the plant fermented liquid and/or the acetic acid bacteria fermented product of the plant fermented liquid exhibited significant effects in improving skin wrinkles and enhancing elasticity, etc. (Table 2).

As used herein, the term “skin soothing” refers to alleviating and relieving erythema, itchiness, or irritated skin areas, etc., and may include alleviation of skin irritation. For example, the skin soothing may include, but is not limited to, alleviating skin itchiness, alleviating skin pain, reducing transdermal water loss and/or reducing redness.

As used herein, the term “inflammation” refers to a kind of in vivo response to damage or infection of a specific tissue, and the main mediator is immune cells. The purpose of such inflammation is to suppress tissue damage as much as possible, remove infectious agents, and regenerate tissue. In the present invention, skin soothing and regeneration may be caused by inhibition of NO production, but is not limited thereto.

As used herein, the term “NO (nitric oxide)” plays various roles such as removing bacteria and tumors, regulating blood pressure, or mediating nerve transmission, etc. However, when an inflammatory reaction occurs, the expression of iNOS (inducible nitric oxide synthase) increases in related cells, resulting in the excess production of NO. Further, the excess production of NO causes tissue damage, genetic mutation, nerve damage, etc., and increases vascular permeability, thereby promoting inflammatory responses such as edema. While NO production is increased in inflamed cells, NO production is suppressed when the skin is soothed, and there exists an essential correlation therebetween.

In one embodiment of the present invention, it was confirmed that the plant fermented liquid and/or the acetic acid bacteria fermented product of the plant fermented liquid also showed excellent NO production inhibitory ability even with a small amount as compared to normal cells, thereby exhibiting skin soothing and inflammation improving effects (Table 3).

As used herein, the term “skin moisturization” refers to all action that maintains the flexibility of the skin by supplying moisture to the skin or blocking evaporation of moisture from the skin and maintains a smooth surface by inducing uniform exfoliation of dead skin cells, and the term “dryness” refers to a state in which the skin is not sufficiently moisturized. The skin moisturization effect may help improve wrinkles and increase elasticity of the skin. Specifically, the skin moisturization may be caused by increasing hyaluronic acid production, but is not limited thereto.

As used herein, the term “hyaluronic acid” refers to a type of glycosaminoglycan and is a chain-shaped polymeric polysaccharide in which glucuronic acid and N-acetylglucosamine residues are repeatedly linked. Hyaluronic acid can contain water corresponding to 300 to 1000 times its own weight, and exhibits a moisturizing effect on the skin by binding with moisture.

In one embodiment of the present invention, it was confirmed that the plant fermented liquid and/or the acetic acid bacteria fermented product of the plant fermented liquid had an excellent hyaluronic acid synthesis enhancement rate, and based on the result, it was confirmed that the plant fermented liquid and/or the acetic acid bacteria fermented product of the plant fermented liquid exhibited significant effects in skin moisturization, etc.

As used herein, the term “whitening” comprehensively includes a method of increasing the brightness of the skin whose brightness has decreased due to an excess of pigments such as melanin, or maintaining the brightness of the skin at a certain level, and the skin with increased brightness formed by the above method, and may specifically mean skin whitening. The “skin whitening” may be understood as an improvement of symptoms, such as melasma and freckles, caused by an increase in melanin by inhibition of melanin production as tyrosinase activity is inhibited.

In one embodiment of the present invention, it was confirmed that the plant fermented liquid and/or the acetic acid bacteria fermented product of the plant fermented liquid showed an excellent melanin production inhibition rate, thereby exhibiting excellent skin whitening effect.

As used herein, the term “cosmetic composition” may be prepared in the form selected from the group consisting of as solution, ointment for external use, cream, foam, nutritive cosmetic water, softening cosmetic water, perfume, facial mask, softening water, emulsion, makeup base, essence, soap, liquid washing agent, bath foam, sunscreen cream, sun oil, suspension, emulsion, paste, gel, lotion, powder, soap, surfactant-containing cleanser, oil, powder foundation, emulsion foundation, wax foundation, patch and spray, but is not limited thereto.

Additionally, the cosmetic composition of the present invention may further include one or more cosmetically acceptable carrier mixed to general skin cosmetics. As common ingredients, for example, oil, water, surfactants, moisturizers, lower alcohols, thickening agents, chelating agents, colorings, preservatives, fragrances, etc. may be appropriately mixed, but are not limited thereto.

The cosmetically acceptable carrier included in the cosmetic composition of the present invention may vary depending on the formulations.

When the formulation of the cosmetics of the present invention is an ointment, paste, cream or gel, animal oil, vegetable oil, wax, paraffin, starch, tragacanth, cellulose derivatives, polyethylene glycol, silicone, bentonite, silica, talc, zinc oxide, etc., may be used as a carrier ingredient, but the carrier ingredient is not limited thereto. These may be used alone or in a mixture of two or more thereof.

When the formulation of the cosmetics of the present invention is a solution or emulsion, solvents, solubilizing agents or emulsifying agents may be used as a carrier ingredient, and for example, water, ethanol, isopropanol, ethyl carbonate, ethyl acetate, benzyl alcohol, benzyl benzoate, propylene glycol, 1,3-butyl glycol oil, may be used. In particular, cottonseed oil, peanut oil, maize germ oil, olive oil, castor oil and sesame seed oil, glycerol aliphatic ester, polyethylene glycol or aliphatic ester of sorbitan may be used, but the carrier ingredient is not limited thereto. These may be used alone or in a mixture of two or more thereof.

When the formulation of the cosmetics of the present invention is a suspension, liquid diluents such as water, ethanol or propylene glycol, suspending agent, such as ethoxylated isostearyl alcohol, polyoxyethylene sorbitol ester and polyoxyethylene sorbitan ester, microcrystalline cellulose, aluminum metahydroxide, bentonite, agar, or tragacanth may be used as a carrier ingredient. These may be used alone or in a mixture of two or more thereof.

When the formulation of the cosmetics of the present invention is a soap, alkali metal salts of fatty acids, salts of fatty acid hemiesters, fatty acid protein hydrolysates, isethionate, lanolin derivatives, aliphatic alcohol, vegetable oil, glycerol, sugars, etc. may be used as a carrier ingredient, but the carrier ingredient is not limited thereto. These may be used alone or in a mixture of two or more thereof.

When the formulation of the cosmetics of the present invention is a powder or spray, lactose, talc, silica, aluminum hydroxide, calcium silicate, polyamide powder, or a mixture thereof may be used as a carrier ingredient, and in particular, when it is a spray, a propellant such as chlorofluorohydrocarbon, propane/butane or dimethyl ether may be additionally included.

Meanwhile, all components described in the present invention may be preferably included in the composition of the present invention within a range that does not exceed the maximum amount stipulated in the Regulations on the Safety Standards, etc., of Cosmetics of Korea and in the “Safety and Technical Standards for Cosmetics” set by the Chinese government.

Yet another aspect of the present invention provides a quasi-drug composition for improving skin, including any one or more among the Gluconobacter thailandicus strain deposited under Accession No. KCTC 15086BP, a culture of the strain, and a fermented product of the strain.

Even another aspect of the present invention provides a quasi-drug composition for improving skin, including plant fermented liquid or an acetic acid bacterial strain of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger, specifically, plant fermented liquid which is additionally fermented by the Gluconobacter thailandicus strain deposited under Accession No. KCTC 15086BP, a culture of the strain, a fermented product of the strain, or a combination thereof, as an active ingredient.

The “Gluconobacter thailandicus”, “strain”, “culture”, “fermented product”, “plant fermented liquid”, “skin improvement”, “improvement”, “wrinkles”, “elasticity”, “aging”, “collagen”, “skin soothing”, “inflammation”, “NO”, “skin moisturization”, “hyaluronic acid” and “whitening” are as described above.

As used herein, the term “quasi-drug” may be one selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but is not limited thereto.

The quasi-drug composition of the present invention may further include a pharmaceutically acceptable carrier, excipient, or diluent, if necessary, in addition to the above ingredients. The pharmaceutically acceptable carrier, excipient, or diluent is not limited as long as it does not impair the effect of the present invention and may include a filler, an extender, a binder, a wetting agent, a disintegrant, a surfactant, a lubricant, a sweetener, an aromatic, a preservative, etc.

Typical examples of the pharmaceutically acceptable carrier, excipient, and diluent of the present invention include lactose, dextrose, sucrose, sorbitol, mannitol, xylitol, maltitol, starch, gelatin, glycerin, acacia gum, alginate, calcium phosphate, calcium carbonate, calcium silicate, cellulose, methyl cellulose, microcrystalline cellulose, polyvinyl pyrrolidone, water, methyl hydroxybenzoate, propyl hydroxybeonzoate, talc, magnesium stearate, mineral oil, propylene glycol, polyethylene glycol, vegetable oil, injectable ester, witepsol, macrogol, tween 61, cacao butter, laurin butter, etc.

When the plant fermented liquid of the present invention, the strain, a culture of the strain, or a fermented product thereof is used as a quasi-drug, it may further contain one or more active ingredients exhibiting the same or similar functions. For example, it may contain ingredients capable of increasing skin beneficial bacteria, inhibiting skin harmful bacteria, skin soothing, improving skin inflammation, skin whitening, skin regeneration, and wound-healing known in the art. When an additional ingredient capable of improving skin wrinkles, skin whitening, improving skin problems, or skin moisturization, the wrinkle improving effect, elasticity improving effect, skin soothing effect, skin inflammation improving effect, skin moisturization effect, and whitening effect of the composition of the present invention may be further enhanced. When the above additional ingredients are added, skin safety, easiness of formulation, and stability of effective ingredients may be considered according to the combined use.

The quasi-drug composition may further include one or two or more ingredients selected from the group consisting of retinoic acid, TGF, an animal placenta-derived protein, betulinic acid, and a chlorella extract; as an elasticity enhancing ingredient, wrinkle-improving ingredient or moisturizing ingredient known in the art; non-steroid such as flufenamic acid, ibuprofen, benzydamine, indomethacin, prednisolone, dexamethasone, allantoin, azulene, hydrocortisone as an anti-inflammatory ingredient known in the art; and derivatives thereof and various plant extracts. The additional ingredients may be contained in an amount of 0.0001 to 10% by weight based on the total weight of the composition, and the content range may be adjusted according to the requirements such as skin safety, easiness when formulating the plant fermented liquid of the present invention, the strain, a culture or fermented product of the strain, etc.

The formulation methods, doses, usages, components, etc. of quasi-drugs may be appropriately selected from conventional techniques known in the art.

Further another aspect of the present invention provides a food composition for improving skin, including any one or more among the Gluconobacter thailandicus strain deposited under Accession No. KCTC 15086BP, a culture of the strain, and a fermented product of the strain.

Still further another aspect of the present invention provides a food composition for improving skin, including plant fermented liquid or an acetic acid bacterial strain of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger, specifically, plant fermented liquid which is additionally fermented by the Gluconobacter thailandicus strain deposited under Accession No. KCTC 15086BP, a culture of the strain, a fermented product of the strain, or a combination thereof, as an active ingredient.

The “Gluconobacter thailandicus”, “strain”, “culture”, “fermented product”, “plant fermented liquid”, “skin improvement”, “improvement”, “wrinkles”, “elasticity”, “aging”, “collagen”, “skin soothing”, “inflammation”, “NO”, “skin moisturization”, “hyaluronic acid” and “whitening” are as described above.

As used herein, the term “food” may include all foods in a conventional sense, such as meats, sausages, breads, chocolates, candies, snacks, cookies, pizzas, ramens, other noodles, gums, dairy products including ice cream, various kinds of soups, beverages, teas, drinks, alcoholic beverages, vitamin complexes, health functional foods, etc., and is not limited as long as it can include the plant fermented liquid of the present invention, the Gluconobacter thailandicus strain deposited under Accession No. KCTC 15086BP, a culture of the strain, and a fermented product of the strain. Additionally, the food composition may be added to extracted juice, teas, jellies, juices, etc. which were prepared by the plant fermented liquid, the strain, a culture of the strain, and a fermented product of the strain according to the present invention as the main ingredient, and may be prepared in the form of pills, powders, granules, tablets, capsules, or liquids.

As used herein, the term “health functional food”, which has the same meaning as the term “food for special health use (FoSHU)”, refers to a food with high effects in medicinal and medical treatment, processed so as to efficiently exhibit a body modulating function as well as to provide nutrients. In particular, the term “functionality” refers to controlling nutrients for the structure of functions of the human body or obtaining useful effects for hygienic purposes, such as physiological effects, etc. The food of the present invention may be prepared according to a method commonly employed in the art, and raw materials and ingredients commonly used in the art may be added when preparing the food. Additionally, the formulation of the food is not limited so long as it is recognized as a food. The food composition of the present invention may be prepared in various formulations, and the food composition of the present invention uses a food as a raw material unlike generic drugs, and thus has no side effects that may occur during long-term administration of the drugs, is highly portable, and may be administered as an adjuvant for enhancing the immune-boosting effects.

The health food refers to a food that has an active health maintenance or promotion effect compared to general food, and a health supplement food refers to a food intended for health supplement. In some cases, the terms functional food, health food, and health supplement food are used interchangeably.

Specifically, the health functional food is a food prepared by adding any one or more among the plant fermented liquid, the Gluconobacter thailandicus strain deposited under Accession No. KCTC 15086BP, a culture of the strain, and a fermented product of the strain to food materials such as beverages, teas, spices, gum, and confectionery, etc., or a food prepared by means of encapsulation, powdering, or suspension. When the health functional food is ingested, a specific health effect may be obtained. However, unlike general drugs, since the health functional food uses food as a raw material, there is an advantage in that there are no side effects that may occur during long-term administration of the drugs.

The composition may further include a physiologically acceptable carrier, and the type of carrier is not particularly limited, and any carrier commonly used in the art may be used.

Additionally, the composition may contain additional ingredients that are conventionally used in food compositions so as to improve smell, taste, vision, etc. For example, the composition may contain vitamins A, C, D, E, B1, B2, B6, and B12, niacin, biotin, folate, pantothenic acid, etc. In addition, the composition may contain minerals such as zinc (Zn), iron (Fe), calcium (Ca), chromium (Cr), magnesium (Mg), manganese (Mn), copper (Cu), and chromium (Cr), etc. Further, the composition may contain amino acids such as lysine, tryptophan, cysteine, valine, etc.

Additionally, the composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), coloring agents (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-hydrogen tartrate, etc.), fortifiers, emulsifiers, thickeners (adhesive pastes), film-forming agents, gum base agents, antifoaming agents, solvents, improvers, etc. The additives may be selected and used in an appropriate amount according to the food types.

Any one or more among the Gluconobacter thailandicus strain deposited under Accession No. KCTC 15086BP, a culture of the strain, and a fermented product of the strain may be added as it is or used in combination with other foods or food ingredients, and may be appropriately used according to a conventional method. The amount of mixed active ingredients may appropriately be determined depending on the purpose of use (prevention, health, or therapeutic treatment). In general, the food composition of the present invention may be added in an amount of 50 parts by weight or less, specifically 20 parts by weight or less, based on the food or beverage when preparing food or beverage. However, in the case of long-term administration for health and hygiene purposes, the active ingredients may be contained in an amount less than the above-described range, and because there is no problem in terms of safety, the active ingredients may be contained in an amount greater than the above-described range.

In one example, the food composition of the present invention may be used as a health beverage composition, and in such case, the food composition may further contain, as additional components, various flavoring agents or natural carbohydrates, as in conventional drinks. The aforementioned natural carbohydrates may include monosaccharides such as glucose, fructose, etc.; disaccharides such as maltose, sucrose, etc.; polysaccharides such as dextrin, cyclodextrin, etc.; and sugar alcohols such as xylitol, sorbitol, erythritol, etc. Natural sweetening agents such as thaumatin, a stevia extract, etc.; and synthetic sweetening agents such as saccharin, aspartame, etc. may be used as the sweetening agent. A ratio of the additional components may be in a range of 0.01 to 0.04 parts by weight, specifically 0.02 to 0.03 parts by weight based on 100 mL of the composition of the present invention.

In addition to the aforementioned components, the health beverage composition may contain various nutritional supplements, vitamins, electrolytes, flavoring agents, coloring agents, pectic acid and salts thereof, alginic acid and salts thereof, organic acids, protective colloidal thickening agents, pH controlling agents, stabilizing agents, preservatives, glycerin, alcohols, or carbonating agents, etc. Moreover, the composition may contain pulp for preparing a natural fruit juice, a fruit juice drink or a vegetable drink. These ingredients may be used independently or in a mixture. The ratio of such additives is not important, but is generally chosen in a range of 0.01 to 0.1 parts by weight, based on 100 parts by weight of the composition of the present invention.

Specifically, any one or more among the plant fermented liquid, the Gluconobacter thailandicus strain deposited under Accession No. KCTC 15086BP, a culture of the strain, and a fermented product of the strain may be contained in an amount of in 0.00001% to 100% by weight based on the total weight of the food composition.

Still further another aspect of the present invention provides a method for preparing a plant fermented product, including: fermenting plant fermented liquid by the Gluconobacter thailandicus strain deposited under Accession No. KCTC 15086BP.

The “Gluconobacter thailandicus”, “strain”, and “plant fermented liquid” are as described above.

In the present invention, fermentation may be performed by inoculating an acetic acid bacterial strain, specifically, Gluconobacter thailandicus KCTC 15086BP, into the primarily fermented plant fermented liquid, followed by additional fermentation (secondary fermentation) for 3 to 10 days, specifically 5 to 8 days, but is not limited thereto. The acetic acid bacterial strain of the present invention may further include culturing in a medium before inoculation into the plant fermented liquid, but is not limited thereto.

Still further another aspect of the preset invention provides a method for preparing a plant fermented product, including: fermenting plant fermented liquid by an acetic acid bacterial strain, a culture of the strain, a fermented product of the strain, or a combination thereof.

The “acetic acid bacterial strain”, “culture”, and “fermented product” are as described above.

Still further another aspect of the present invention provides a plant fermented product prepared by the above method.

The “plant fermented product” is as described above.

Still further another aspect of the present invention provides a method for improving skin, including: applying a composition containing any one or more among the Gluconobacter thailandicus strain deposited under Accession No. KCTC 15086BP, a culture of the strain, and a fermented product of the strain to a subject.

The “Gluconobacter thailandicus”, “culture”, and “fermented product”, and “skin improvement” are as described above.

As used herein, the term “subject” refers to all animals including humans, and the animals may be not only humans but also mammals such as cattle, horses, sheep, pigs, goats, camels, antelopes, dogs, cats, etc. in need of treatment of the similar symptoms, but are not limited thereto.

Specifically, the “subject” of the present invention may refer to all animals including humans which need wrinkle improvement, skin elasticity improvement, skin inflammation improvement, skin soothing, skin moisturization, or skin whitening or which have insufficient collagen or hyaluronic acid, excessive production of NO, or excessive expression of melanin, and the animals may be not only humans but also mammals such as cows, horses, sheep, pigs, goats, camels, antelopes, dogs, cats, etc. in need of treatment of similar symptoms, but are not limited thereto.

As used herein, the term “applying” refers to any method of contacting the skin of an individual with the composition according to the present disclosure by any suitable method, and through this, it is intended to absorb the corresponding composition into the skin.

When the composition of the present disclosure is applied to the wrinkle improvement, skin elasticity improvement, skin inflammation improvement, skin soothing, skin moisturization or skin whitening effects, and the method may include applying the composition to the individual in an amount effective to exhibit the effects.

Still further another aspect of the present invention provides a method for improving skin, including: applying a composition containing plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient to a subject.

The “plant fermented liquid”, and “skin improvement” are as described above.

### Advantageous Effects

Since the plant fermented liquid of the present invention, the Gluconobacter thailandicus strain, a culture of the strain, or a fermented product of the strain has an excellent skin improvement effect, it can be widely used as cosmetic compositions, quasi-drug compositions, and food compositions for skin improvement. Accordingly, various industrial applications such as cosmetics, quasi-drugs, and foods can be expected.

### DETAILED DESCRIPTION OF EMBODIMENTS

The present invention will be described in more detail by way of Examples and Experimental Examples. However, these Examples and Experimental Examples are given for illustrative purposes only, and the scope of the invention is not intended to be limited by these Examples and Experimental Examples.

### Example 1. Preparation of Plant Fermented Liquid

Fifty-five plants shown in Table 1 were selected, and the plants were pickled and aged with sugar extracted from sugar beet from Hokkaido. During the aging process, plant extract is released to the outside by osmotic pressure, and the released extract is fermented by natural microorganisms attached to the plant (sugar osmosis extraction method). The fermentation is carried out for at least 10 years in the natural environment of Hokkaido with an average temperature of 20° C. to 30° C. and humidity of about 60% to 70%, through which plant fermented liquid was prepared.

TABLE 1

| No. | Plant |
|---|---|
| 1 | Okra |
| 2 | Kiwi |
| 3 | Onion |
| 4 | Green onion |
| 5 | Garlic |
| 6 | Garlic chive |
| 7 | Wild celery |
| 8 | Celery |
| 9 | Burdock root |
| 10 | Asparagus |
| 11 | Cauliflower |
| 12 | Cabbage |
| 13 | Bok choy |
| 14 | Chinese cabbage |
| 15 | Japanese mustard spinach |
| 16 | Red pepper |
| 17 | Paprika |
| 18 | Parsley |
| 19 | Crown daisy |
| 20 | Chrysanthemum |
| 21 | Watermelon |
| 22 | Lemon |
| 23 | Tangerine |
| 24 | East Asian wild parsley |
| 25 | Melon |
| 26 | Cucumber |
| 27 | Pumpkin |
| 28 | Carrot |
| 29 | Persimmon |
| 30 | Enoki mushroom |
| 31 | Bean sprout |
| 32 | Sweet potato |
| 33 | Head lettuce |
| 34 | Leaf lettuce |
| 35 | Shiitake mushroom |
| 36 | Kelp |
| 37 | Banana |
| 38 | Watercress |
| 39 | Lotus root |
| 40 | Perilla leaf |
| 41 | Avocado |
| 42 | Japanese apricot |
| 43 | Peach |
| 44 | Plum |
| 45 | Pear |
| 46 | Apple |
| 47 | Radish |
| 48 | Tomato |
| 49 | Eggplant |
| 50 | Potato |
| 51 | Spinach |
| 52 | Grape |
| 53 | Wasabi |
| 54 | Myoga |
| 55 | Ginger |

### Example 2. Preparation of Acetic Acid Bacteria (Gluconobacter thailandicus NV1) Fermented Product of Plant Fermented Liquid

### Example 2-1. Isolation of Acetic Acid Bacteria (Gluconobacter thailandicus NV1) Derived from Japanese Apricot Enzyme Solution

Gluconobacter thailandicus NV1 strain, an acetic acid bacterium derived from Japanese apricot enzyme solution, was obtained.

The Gluconobacter thailandicus NV1 strain was deposited at the Korean Collection for Type Cultures (KCTC) of Korea Research Institute of Bioscience and Biotechnology under Budapest Treaty on Sep. 20, 2022, with Accession No. KCTC 15086BP. The strain was confirmed to have the 16S rRNA nucleotide sequence of SEQ ID NO: 1.

### Example 2-2. Preparation of Acetic Acid Bacteria Fermented Product of Plant Fermented Liquid

The Gluconobacter thailandicus NV1 strain was inoculated into a YPM medium and incubated at about 30° C. for 2 days. Thereafter, the acetic acid bacterial strain culture was inoculated into the plant fermented liquid of Example 1, and stationary fermentation, i.e., acetic acid fermentation (secondary fermentation), was carried out at about 30° C. and 60% humidity for 7 days.

### Example 3. Confirmation of Wrinkle Improvement and Elasticity Improvement Effects of Plant Fermented Liquid and Acetic Acid Bacteria (Gluconobacter thailandicus NV1) Fermented Product of Plant Fermented Liquid

Samples were treated with human dermal fibroblast for 48 hours at each concentration. The collagen synthesis rate was measured using the Procollagen Type I C-peptide (PIP) EIA Kit. The collagen synthesis efficacy of the samples was calculated and measured as values in comparison with the negative control group (DMEM containing 0% serum).

In order to confirm the wrinkle improvement and elasticity improvement effects of 1-year-old plant fermented liquid, 10-year-old or older plant fermented liquid, and acetic acid bacteria (Gluconobacter thailandicus NV1) fermented product of plant fermented liquid, the collagen synthesis rates were compared upon treatment of normal cells, 1-year-old plant fermented liquid, 10-year-old or older plant fermented liquid, and acetic acid bacteria fermented product of plant fermented liquid (Table 2).

TABLE 2

| | Amount Exhibiting Efficacy |
|---|---|
| Sample | (Collagen synthesis rate) |
| Normal cells | 0 |
| (Negative control) | |
| 1-year-old plant | 1% (14.94%) |
| fermented liquid | |
| 10-year-old or older plant | 0.001% (10.7%) |
| fermented liquid | |
| Acetic acid fermented product | 0.000001% (11.77%) |
| of 10-year-old or older plant | |
| fermented liquid | |

As shown in the results of Table 2, it was confirmed that the 10-year-old or older plant fermented liquid exhibited collagen synthesis ability at a concentration of 1/1000 (0.001%, 1 ppm=10000 ppb) compared to the 1-year-old plant fermented liquid, and the acetic acid bacteria fermented product exhibited collagen synthesis ability at a concentration of 1/1000000 compared to the 1-year-old plant fermented liquid and 1/1000 compared to the 10-year-old or older plant fermented liquid (0.000001%, 0.01 ppm=10 ppb).

Accordingly, it was found that the plant fermented liquid and the acetic acid bacteria fermented product of the plant fermented liquid of the present invention exhibited excellent effects such as improving skin wrinkles and enhancing elasticity, etc.

### Example 4. Confirmation of Skin Soothing and Inflammation Improvement Effects of Plant Fermented Liquid and Acetic Acid Bacteria (Gluconobacter thailandicus NV1) Fermented Product of Plant Fermented Liquid

Raw 264.7 cells were diluted for each concentration, treated with 1 μg/ml of LPS, and incubated for 24 hours. The NO production inhibitory ability was evaluated as follows using the NO assay kit.

NO production inhibition ability (%)=(1−(NO production amount when sample is added/NO production amount when sample is not added)×100

In order to confirm the skin soothing and inflammation improvement effects of the acetic acid bacteria (Gluconobacter thailandicus NV1) fermented product of the plant fermented liquid, the inhibitory ability of NO production was compared for normal cells, and when the normal cells were treated with L-NMMA and acetic acid bacteria fermented product of the plant fermented liquid (Table 3).

TABLE 3

| | Inhibitory Ability of |
|---|---|
| | NO Production |
| Sample | (% of control) |
| Normal cells | 0 |
| (Negative control) | |
| Normal cells + L-NMMA | 64.6 |
| (Positive control, | |
| 20 μg/mL) | |
| Acetic acid fermented product | 28.5 |
| of 10-year-old or older | |
| fermented liquid (0.01%) | |
| Acetic acid fermented product | 21.2 |
| of 10-year-old or older | |
| fermented liquid (0.1%) | |
| Acetic acid fermented product | 29.2 |
| of 10-year-old or older | |
| fermented liquid (1%) | |

As shown in the results of Table 3, it was confirmed that the NO production inhibitory ability increased when normal cells were treated with the acetic acid bacteria fermented product.

Accordingly, it was found that the plant fermented liquid and the acetic acid bacteria fermented product of the plant fermented liquid of the present invention exhibited excellent effects such as skin soothing and inflammation improvement, etc.

### Example 5. Confirmation of Skin Moisturization Effect of Plant Fermented Liquid and Acetic Acid Bacteria (Gluconobacter thailandicus NV1) Fermented Product of Plant Fermented Liquid

Samples were treated with human keratinocyte HaCaT for 72 hours at each concentration. Hyaluronic acid synthesis was compared with the control group (DMEM treatment containing 0% serum) using Quantikine ELISA Hyaluronan from R&D Systems to quantify the moisturizing ability.

TABLE 4

| | Hyaluronic Acid |
|---|---|
| | Synthesis Ability |
| Sample | (% of control) |
| Normal cells (Negative control) | 0 |
| Normal cells + Retinoic acid | 18.6 |
| (Positive control, 30 ppb) | |
| Acetic acid fermented product | 4.3% |
| of 10-year-old or older | |
| fermented liquid (1 ppm) | |

Accordingly, it was found that the plant fermented liquid and the acetic acid bacteria fermented product of the plant fermented liquid of the present invention exhibited excellent effects such as moisturizing and soothing the skin, etc.

### Example 6. Confirmation of Skin Whitening Effect of Plant Fermented Liquid and Acetic Acid Bacteria (Gluconobacter thailandicus NV1) Fermented Product of Plant Fermented Liquid

Samples were diluted in B16f10 melanocytes at each concentration, treated for 72 hours, and incubated, and then, the amount of melanin pigment present in melanocytes was quantified.

The melanin content was calculated in comparison to the total protein amount, and the values thereof were measured as percentage (%) based on the DMSO control (DMEM containing 0% serum) set to 100%.

TABLE 5

| | Inhibition of |
|---|---|
| | Melanin Synthesis |
| Sample | (% of control) |
| Normal cells (Negative control) | 0 |
| Normal cells + Arbutin | 13.3 |
| (Positive control, 200 ppm) | |
| 10-year-old or older fermented | 15.9% |
| liquid (0.1%) | |
| Acetic acid fermented product | 17.7% |
| of 10-year-old or older | |
| fermented liquid (0.1%) | |

As shown in the results of Table 5, it was confirmed that the 10-year-old or older fermented liquid and the acetic acid bacteria fermented product showed excellent melanin synthesis inhibition rate than the positive control group.

Accordingly, it was found that the plant fermented liquid and the acetic acid bacteria fermented product of the plant fermented liquid of the present invention exhibited excellent effects on skin whitening.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.
[Deposition No.]
Depository Institution: Korean Collection for Type Cultures
Accession No.: KCTC 15086BP
Deposition Date: 20220920

1. A method for producing a plant fermented product, comprising: fermenting plant fermented liquid by an acetic acid bacterial strain, a culture of the strain, a fermented product of the strain, or a combination thereof.

2. The method of claim 1, wherein the acetic acid bacterial strain is a Gluconobacter thailandicus strain deposited under Accession No. KCTC 15086BP.

3. The method of claim 1, wherein the plant fermented liquid is a fermented product of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger.

4. The method of claim 1, wherein the plant fermented liquid is fermented for 5 years or more.

5. A plant fermented product produced by the method of claim 1.

6. A composition comprising the plant fermented product of claim 5.

7. A method for improving skin, comprising: applying a composition containing any one or more among the Gluconobacter thailandicus strain deposited under Accession No. KCTC 15086BP, a culture of the strain, and a fermented product of the strain to a subject.

8. A method for improving skin, comprising: applying a composition containing plant fermented liquid of okra, kiwi, onion, green onion, garlic, garlic chive, wild celery, celery, burdock root, asparagus, cauliflower, cabbage, bok choy, Chinese cabbage, Japanese mustard spinach, red pepper, paprika, parsley, crown daisy, chrysanthemum, watermelon, lemon, tangerine, East Asian wild parsley, melon, cucumber, pumpkin, carrot, persimmon, enoki mushroom, bean sprout, sweet potato, head lettuce, leaf lettuce, shiitake mushroom, kelp, banana, watercress, lotus root, perilla leaf, avocado, Japanese apricot, peach, plum, pear, apple, radish, tomato, eggplant, potato, spinach, grape, wasabi, myoga, and ginger as an active ingredient to a subject.

9. The method of claim 8, wherein the plant fermented liquid is fermented for 5 years or more.

10. The method of claim 8, wherein the skin improvement is any one or more selected from the group consisting of wrinkle improvement, skin elasticity improvement, skin inflammation improvement, skin soothing, skin moisturization, and skin whitening.

11. The method of claim 8, wherein the composition has the effects of promoting collagen synthesis, inhibiting NO production, promoting hyaluronic acid synthesis, or inhibiting melanin expression.

12. The method of claim 8, wherein the plant fermented liquid is fermented by an acetic acid bacterial strain, a culture of the strain, a fermented product of the strain, or a combination thereof.

13. The method of claim 12, wherein the acetic acid bacterial strain is a Gluconobacter thailandicus strain deposited under Accession No. KCTC 15086BP.


SEQUENCE LISTING (1 sequence)

SEQ ID NO: 1 - RNA, 1104 nt, unidentified
  source 1..1104: mol_type = genomic RNA
tggctcagagcgaacgctggcggcatgcttaacacatgcaagtcgcacggatctttcgggattagtggcggacgggtgagtaacgcgtagggatctatccacgggtgggggacaactccgggaaactggagctaataccgcatgatacctgagggtcaaaggcgcaagtcgcctgtggaggaacctgcgttcgattagctagttggtggggtaaaggcctaccaaggcgatgatcgatagctggtttgagaggatgatcagccacactgggactgagacacggcccagactcctacgggaggcagcagtggggaatattggacaatgggcgaaagcctgatccagcaatgccgcgtgtgtgaagaaggtcttcggattgtaaagcactttcgacggggacgatgatgacggtacccgtagaagaagccccggctaacttcgtgccagcagccgcggtaatacgaagggggctagcgttgctcggaatgactgggcgtaaagggcgcgtaggcggttgatgcagtcagatgtgaaatccccgggcttaacctgggaactgcatttgagacgcattgactagaggtcgagagagggttgtggaattcccagtgtagaggtgaaattcgtagatattgggaagaacaccggtggcgaaggcggcaacctggctcgatactgacgctgaggcgcgaaagcgtggggagcaaacaggattagataccctggtagtccacgctgtaaacgatgtgtgctggatgttgggtaacttagttactcagtgtcgaagctaacgcgctaagcacaccgcctggggagtacggccgcaaggttgaaactcaaaggaattgacgggggcccgcacaagcggtggagcatgtggtttaattcgaagcaacgcgcagaaccttaccagggcttgcatggggaggacgtactcagagatgggtatttcttcggacctcccgcacaggtgctgcatggctgtcgtcagctcgtgtcgtgagatgttgggttaagtcccgcaacgagcgcaacccttgtctttagttgccagcactttcaggtgggcactctagagagactgccggtgacaagccggaggaa